(19) United States
(12) Patent Application Publication
(10) Pub. No.: US 2018/0035945 A1
(43) Pub. Date: Feb. 8, 2018

(54) SMART COLLAR AND SYSTEM THEREOF

(71) Applicant: Yi-Sen HSIEH, Taoyuan City (TW)

(72) Inventor: Yi-Sen HSIEH, Taoyuan City (TW)

(21) Appl. No.: 15/668,100

(22) Filed: Aug. 3, 2017

(30) Foreign Application Priority Data
Aug. 3, 2016 (TW) 105124679

(51) Int. Cl. A61B 5/00; A61B 5/0205; A61B 5/1455; A61B 5/11

(52) U.S. Cl. CPC A61B 5/6802; A61B 5/11; A61B 5/0015; A61B 5/742; A61B 5/02055; A61B 5/14551; A61B 5/4806; A61B 5/7278; A61B 2560/0214; A61B 2562/0219; A61B 5/02427

(57) ABSTRACT

A smart collar includes a main body, a power module, an image capturing module, a sensing module, a process-storage module, and a wireless transmitting module. The power module includes a power control unit and a battery. The image capturing module retrieves at least one image data. The sensing module includes at least one sensor for detecting the physical condition, motion condition and environment condition of the user so as to obtain physical condition data, motion condition data and environment condition data. The process-storage module includes at least one processing unit and at least one memory unit. The processing unit generates the user condition data according to the physical condition data, the motion condition data and the environment condition data. The memory unit stores the image data and the user condition data. The wireless transmitting module transmits the image data and the user condition data to a peripheral device.

### CROSS REFERENCE TO RELATED APPLICATIONS

This Non-provisional application claims priority under 35 U.S.C. §119(a) on Patent Application No(s). 105124679 filed in Taiwan, Republic of China on Aug. 3, 2016, the entire contents of which are hereby incorporated by reference.

### BACKGROUND

### Technology Field

The present disclosure relates to a smart collar and a smart collar system that can detect the physical condition, motion condition and environment condition of a user.

### Description of Related Art

Recently, the wearable devices become more and more popular in the fields of medical, entertainment, sports, fitness, environment sensing, and the likes. The functions of the wearable devices are rapidly developed, and the wearable devices are designed with multiple functions and applied for different applications. However, the conventional wearable devices still need some improvements. For example, the core components can be manufactured with more compact size and light weight. Besides, the wearable device, which is integrated with the modules and substrates of multiple sensors, may cause a slight discomfort on the user's body. Because the power consumption of the wearable device is large, the device does not have good power capacity, use time and standby time. Moreover, most wearable devices are not equipped with the image capturing module, so they need to connect to the smart phone for utilizing the image capturing module of the smart phone to retrieve the image data. Thus, the user cannot directly utilize the wearable device to record the detailed sport events and retrieve other information. This can cause the inconvenience and function limitation as utilizing the wearable device.

Therefore, it is important to provide a smart collar and a smart collar system that can detect the physical condition, motion condition and environment condition of a user, have good power capacity, use time and standby time, and are equipped with an image capturing module without inconvenience and function limitation in operation.

### SUMMARY

In view of the foregoing, the present disclosure is to provide a smart collar and a smart collar system that can detect the physical condition, motion condition and environment condition of a user, have good power capacity, use time and standby time, and are equipped with an image capturing module without inconvenience and function limitation in operation.

To achieve the above, the present disclosure provides a smart collar configured for detecting a physical condition, a motion condition and an environment condition of a user for retrieving user condition data. The smart collar includes a main body, a power module, an image capturing module, a sensing module, a process-storage module, and a wireless transmitting module. The power module is disposed in the main body and includes a power control unit and a battery. The image capturing module is coupled to the power module and retrieves at least one image data. The sensing module is coupled to the power module and includes at least one sensor for detecting at least one physical condition, at least one motion condition and at least one environment condition of the user so as to obtain physical condition data, motion condition data and environment condition data. The process-storage module is coupled to the image capturing module and the sensing module and includes at least a processing unit and at least a memory unit. The memory unit is coupled to the processing unit, and the processing unit generates the user condition data according to the physical condition data, the motion condition data and the environment condition data. The memory unit stores the image data and the user condition data. The wireless transmitting module is communicated with a peripheral device and transmits the image data and the user condition data to the peripheral device.

To achieve the above, the disclosure also provides a smart collar system including at least a peripheral device and a smart collar. The smart collar is configured for detecting a physical condition, a motion condition and an environment condition of a user. The smart collar includes a main body, a power module, an image capturing module, a sensing module, a process-storage module, and a wireless transmitting module. The power module is disposed in the main body and includes a power control unit and a battery. The image capturing module is coupled to the power module and retrieves at least one image data. The sensing module is coupled to the power module and includes at least a sensor for detecting at least one physical condition, at least one motion condition and at least one environment condition of the user so as to obtain physical condition data, motion condition data and environment condition data. The process-storage module is coupled to the image capturing module and the sensing module and includes at least a processing unit and at least a memory unit. The memory unit is coupled to the processing unit, and the processing unit generates the user condition data according to the physical condition data, the motion condition data and the environment condition data. The memory unit stores the image data and the user condition data. The wireless transmitting module is communicated with the peripheral device and transmits the image data and the user condition data to the peripheral device.

In one embodiment, the smart collar further includes a voice retrieving module coupled to the power module and the process-storage module. The voice retrieving module retrieves at least one voice data, and the storage unit further stores the voice data.

In one embodiment, the smart collar further includes a display module disposed in the main body and coupled to the process-storage module.

In one embodiment, the display module is a lamp display module.

In one embodiment, the smart collar further includes a protection body covering the main body.

In one embodiment, the protection body is made of rubber or silica gel.

In one embodiment, the protection body is an extendable elastic body having an opening, and the opening is disposed outside the main body.

In one embodiment, when the extendable elastic body is stretched, the main body is exposed from the extendable elastic body via the opening, and one or a combination of the power module, the image capturing module, the sensing module and the process-storage module is adjusted to control the smart collar to switch from a first configuration to a second configuration.

In one embodiment, the sensor is one or a combination of an optical heart rate sensor, a reflective pulse oximeter, a multi-axis gyroscope sensor, a multi-axis acceleration sensor, a geomagnetism sensor, an inertial sensor, a thermal sensor, an air pressure sensor, a humidity sensor, a pressure sensor, a height sensor, a light sensor, and a gas sensor.

In one embodiment, the user condition data comprises one or a combination of heart rate information, pulse information, Blood oxygen information, emotion information, sleep information, step information, distance information, caloric consumption information, body information, sitting time information, temperature infoiination, air pressure information, humidity information, pressure information, height information, light information, and gas information.

As mentioned above, the smart collar of the disclosure is configured to detecting the physical condition, motion condition and environment condition of a user so as to retrieve the user condition data. The smart collar includes a main body, a power module, an image capturing module, a sensing module, a process-storage module, and a wireless transmitting module. Thus, the smart collar has good power capacity, use time and standby time, and the user does not need to recharge the smart collar frequently. Besides, the smart collar can record the detailed sport events and retrieve other infoiiiiation.

In addition, the smart collar of the disclosure can transmit the image data and user condition data to the peripheral device through the wireless transmitting module. Moreover, the main body can integrate with all modules, and the smart collar can be bent to fit the human body without affecting the functions of the modules.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1 is a block diagram of a smart collar according to an embodiment of the disclosure;

FIG. 2A is a block diagram of a smart collar according to another embodiment of the disclosure;

FIG. 2B is a schematic diagram of the smart collar of FIG. 2A, which includes a display module;

FIG. 2C is a schematic diagram showing another aspect of the smart collar of FIG. 2B, which includes a display module;

FIG. 3A is a schematic diagram of a smart collar according to another embodiment of the disclosure;

FIG. 3B is a schematic diagram showing another aspect of the smart collar of FIG. 3A;

FIG. 4A is a schematic diagram of a smart collar, which has a protection body, according to another embodiment of the disclosure;

FIG. 4B is a schematic diagram of the smart collar of FIG. 4A as the opening of the protection body is exposed; and

FIG. 5 is a block diagram of a smart collar system according to an embodiment of the disclosure.

### DETAILED DESCRIPTION OF THE DISCLOSURE

The present disclosure will be apparent from the following detailed description, which proceeds with reference to the accompanying drawings, wherein the same references relate to the same elements.

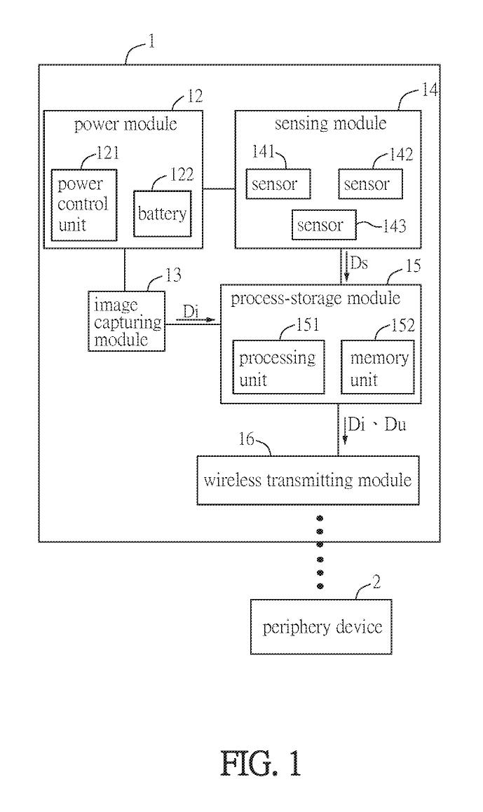

FIG. 1 is a block diagram of a smart collar according to an embodiment of the disclosure. Referring to FIG. 1, a smart collar 1 includes a main body 11 (not shown), a power module 12, an image capturing module 13, a sensing module 14, a process-storage module 15, and a wireless transmitting module 16. The smart collar 1 is configured to detect a physical condition, a motion condition and an environment condition of a user for retrieving user condition data. The smart collar 1 can be made of fashion and elegant materials such as platinum, gold alloy, silver, gold or leather. When the user wears the smart collar 1, the smart collar 1 can detect the physical condition, motion condition and environment condition of the user.

The main body 11 is a printed circuit board. To be noted, the conventional rigid printed circuit board has a rigid property, so it may restrict the size and design of the smart collar 1 and is not suitable for manufacturing the smart collar 1. Preferably, the main body 11 is made of a flexible printed circuit board, so that the user can comfortably wear the smart collar 1, and the smart collar 1 can fit the body of the user. In order to make the smart collar 1 have higher clock and better performance, the main body 11 can be cooperated with multilayer of printed circuit boards having more power layers and ground layers or an HDI (High Density Interconnection) printed circuit board. Accordingly, the main body 11 can have better electric properties, RF optimization and anti EMI ability.

The main body 11 can integrate other modules or components of the smart collar 1 such as the sensing module 14 and the sensors of the sensing module 14, which are manufactured by MEMS (Microelectromechanical systems). In addition, the modules and the main body 11 can be integrated and disposed in the smart collar 1. When the user wears the smart collar 1, it can be bent to fit the shape of the human body, and the functions of the modules and components in the main body 11 are not affected.

The power module 12 includes a power control unit 121 and a battery 122. The power control unit 121 controls the battery 122 to properly output the power to the modules according to the configuration of the modules. For example, the power control unit 121 can control the battery 122 to reduce the power supply to some modules. When the smart collar 1 is not connected to any device, the power supply to the wireless transmitting module can be reduced. When the smart collar 1 is in a standby mode, the power supply to the display module can be reduced. This configuration can improve the power capacity, use time and standby time of the smart ring 1, and the user does not have to frequently charge the smart collar 1.

Since the loading wire voltages and the power operation voltages of the modules in the main body 11 are different, the power control unit 121 further includes a specific DC/DC converter, such as the switching mode inductor-based DC/DC converter or the multitrack DC/DC converter with shared inductance. In order to increase the performance of the smart collar 1 and extend the lifetime of the battery 122, the power control unit 121 may include a switching mode inductor-based DC/DC converter with inbuilt capacitance. Moreover, the battery 122 can be a thin lithium polymer battery, which has small volume and high electricity capacity. This kind of battery can occupy a small space inside the main body 11 and increase the power capacity of the smart collar 1.

The image capturing module 13 can be an image sensor such as a CCD (Charge Coupled Device) or a CMOS (Complementary Metal-Oxide Semiconductor) sensor. The image capturing module 13 is coupled to the power module 12 and retrieves at least one image data Di. The image data Di can be sent to a periphery device 2 via the wireless transmitting module 16, and the periphery device 2 can display the image. The detailed description of the wireless transmitting module 16 will be discussed in the following. In other aspects, the smart collar 1 is connected to the periphery device 2 via a transmission line, and the image data Di is sent from the smart collar 1 to the periphery device 2 via the transmission line. This disclosure is not limited.

In addition, the image capturing module 13 may include an infrared image sensor for retrieving the image data Di. In this case, the retrieved image data Di includes the temperature image data or streaming temperature image data, so that the smart collar 1 can have the function of thermal image camera. Preferably, this image capturing module 13 can be used to record the thermal image of the fat burning status of the user when the user is doing exercise or workout.

In addition, the user may take off the smart collar 1 and use the image capturing module 13 to record the procedure and other information details in the exercise or sport of the user. Of course, the user can still wear the smart collar 1 for retrieving the thermal image of the environment. For example, it is possible to record the theiiiial image data of a group for the reference of preventing disease transmission. Besides, the image data Di can be provided to assist and guide the action of the user when the user is staying in a poor vision situation (e.g. in the night, dark, foggy or fire scene) and dangerous environment. Accordingly, the configuration of the image capturing module 13 can sufficiently improve the function of the smart collar 1.

The sensing module 14 is coupled to the power module 12 and includes at least one sensor. In this embodiment, the sensing module 14 includes three sensors 141, 142 and 143. Of course, in other embodiments, the sensing module 14 may include the sensors of different number, and this disclosure is not limited.

The sensor can detect at least one physical condition, at least one motion condition and at least one environment condition of the user so as to obtain physical condition data, motion condition data and environment condition data. For example, the sensor 141 can be an optical heart rate sensor or a reflective pulse oximeter for detecting the physical condition of the user, the sensor 142 can be a multi-axis gyroscope sensor, a multi-axis acceleration sensor, a geomagnetism sensor or an inertial sensor for detecting the motion condition of the user, and the sensor 143 can be a thermal sensor, an air pressure sensor, a humidity sensor, a pressure sensor, a height sensor, a light sensor or a gas sensor for detecting the environment condition of the user. Accordingly, the smart collar 1 can retrieve the physical condition data, motion condition data and environment condition data of the user by the sensors of the sensing module 14. To make the drawings for clear, the sensing condition data Ds are shown in the figures to present the three condition data retrieved by the sensors 141, 142 and 143.

The process-storage module 15 is coupled to the image capturing module 13 and the sensing module 14, and includes at least a processing unit and at least a memory unit. In this embodiment, the process-storage module 15 includes a processing unit 151 and a memory unit 152. Of course, in other embodiments, the process-storage module 15 may include multiple processing units and multiple memory units, and this disclosure is not limited. The processing unit 151 can be a CPU, a MPU, or controller. The memory unit 152 is coupled to the processing unit 151, and the memory unit 152 can be a non-transitory computer readable storage medium such as a flash memory or SRAM.

The processing unit 151 generates the user condition data Du according to the physical condition data, the motion condition data and the environment condition data, which are retrieved by the sensors 141, 142 and 143. The memory unit 152 stores the image data Di and the user condition data Du. The user condition data Du one or a combination of heart rate information, pulse information, Blood oxygen information, emotion information, sleep information, step information, distance information, caloric consumption information, body information, sitting time information, temperature information, air pressure information, humidity information, pressure information, height information, light information, and gas information.

The wireless transmitting module 16 receives the user condition data Du from the process-storage module 15. In this embodiment, the smart collar 1 is communicated with a peripheral device 2 through the wireless transmitting module 16, and the wireless transmitting module 16 can transmit the user condition data Du to the peripheral device 2. The periphery device 2 can be a smart electronic device having the wireless transmission function (e.g. a smart phone, a tablet computer, a notebook computer, or a remote server). The wireless transmitting module 16 can be a low-power short-distance wireless transmission module such as a low-power Bluetooth module, a ZigBee module, an ANT+module, or a near field communication module. The wireless transmitting module 16 is communicated with the periphery device 2, so that the smart collar 1 can transmit the data retrieved by the sensors to the periphery device 2. Then, the periphery device 2 can perform a complex data analyzing and processing or a large amount of calculation. Thus, the smart collar 1 can be compact and light weight and have lower power consumption. Besides, the smart collar 1 can receive the related information and announcement from the periphery device 2 through the wireless transmitting module 16. For example, the related information and announcement can be a text, a telephone call, the information of friends, or other information and data.

Similarly, the user condition data Du can be transmitted to the periphery device 2 through the wireless transmitting module 16 and then displayed by the periphery device 2. In other aspects, the smart collar 1 is connected to the periphery device 2 via a transmission line, and the user condition data Du is sent from the smart collar 1 to the periphery device 2 for displaying via the transmission line. This disclosure is not limited. The user condition data Du may include any one or combination of the above-mentioned data, and this disclosure is not limited.

In the smart collar of this embodiment, the configuration of the power module can improve the power capacity, use time and standby time of the smart ring, and the user does not have to frequently charge the smart collar. In addition, the user may take off the smart collar and use the image capturing module to record the procedure and other information details in the exercise or sport of the user, or the user can still wear the smart collar for recording the procedure and other information details in the exercise or sport of the user. The configuration of the multiple sensors allows the smart collar to retrieve the detailed physical condition data, motion condition data and environment condition data of the user. The processing unit can generate the user condition data according to the physical condition data, the motion condition data and the environment condition data, which are retrieved by the sensors, and then the user condition data and the image data are stored in the memory unit.

Besides, the smart collar can transmit the image data and the user condition data to the periphery device through the wireless transmitting module. Moreover, the main body can integrate all modules and then disposed in the smart collar. When the user wears the smart collar, it can be bent to fit the shape of the human body, and the functions of the modules and components in the main body are not affected.

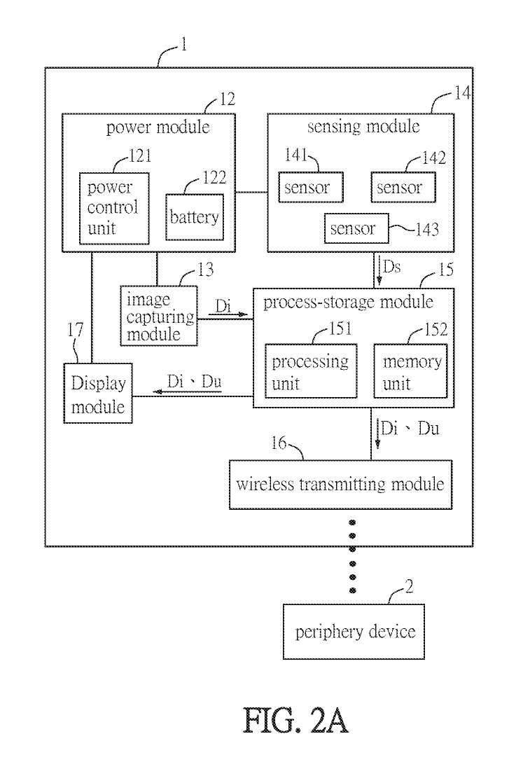

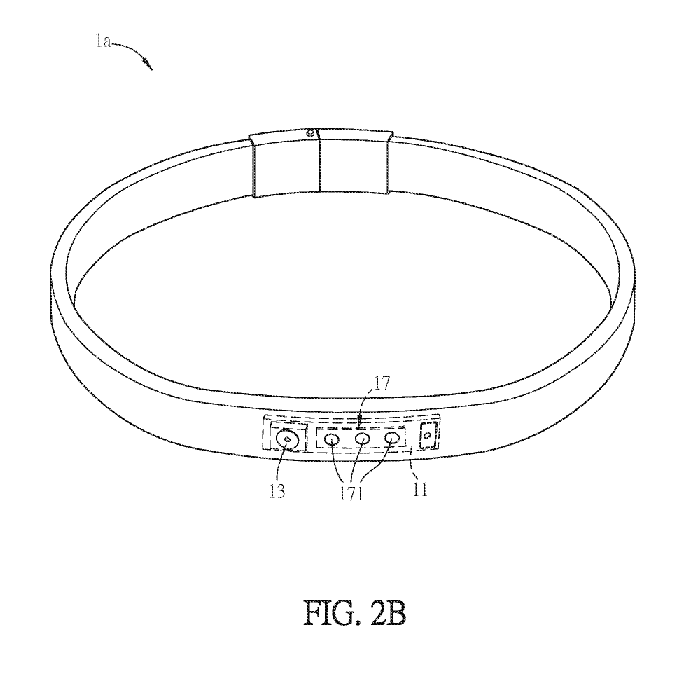

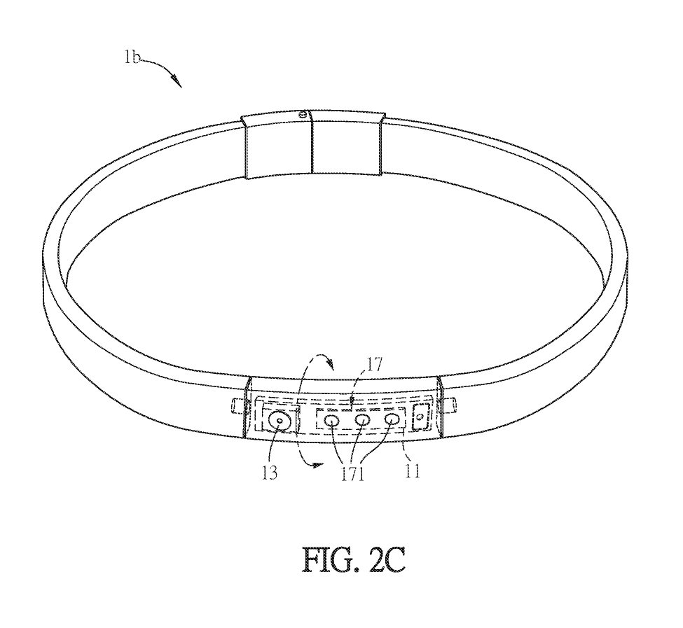

FIG. 2A is a block diagram of a smart collar according to another embodiment of the disclosure, FIG. 2B is a schematic diagram of the smart collar of FIG. 2A, which includes a display module, and FIG. 2C is a schematic diagram showing another aspect of the smart collar of FIG. 2B, which includes a display module.

Referring to FIGS. 2A and 2B, the smart collar 1a includes a main body 11, a power module 12, an image capturing module 13, a sensing module 14, a process-storage module 15, and a wireless transmitting module 16. The smart collar 1a further includes a display module 17, which is disposed in the main body 11 and coupled to the power module 12 and the process-storage module 15.

In this embodiment, the display module 17 is a lamp display module, which includes three indicators 171 for an example. The three indicators 171 can display different colors or the same color, and they can be properly arranged to indicate a plurality of different messages. In other aspects, the display module 171 may have different arrangement. For example, the display module 171 may have four or more indicators or have a display screen, and this disclosure is not limited. In this embodiment, the smart collar 1 can simply show the operation mode or multiple messages by the indicators 171. Compared with other wearable devices having a display screen, the smart collar 1a does not need to frequently display the various user condition infoi nation.

In addition, the smart collar 1a can remind the user to perform the detection of a physical condition, a motion condition and an environment condition by the indicators. For example, the smart collar 1a can remind the user to perform an exercise by three blue indicators 171. In practice, when the user sees that the three indicators 171 all emit blue light, he/she is reminded to start or prepare to do the exercise. In another case, the indicators 171 may flash the red light in a frequency, which matches the detected heart rate or heartbeat, so that the user can intuitionally observe his/her heart rate or heartbeat. In another case, the indicators 171 can show the residual power of the smart collar 1a. For example, the indicator 171 emits green light when the residual power is over 80%, the indicator 171 emits orange light when the residual power is between 20% and 80%, and the indicator 171 flashes red light when the residual power is lower than 20%. In another case, when the smart collar 1a is performing an image capturing function or transmitting data to the periphery device 2, the indicator 171 can emit a different color.

The display and arrangement of the indicators 171 can display the information of the smart collar 1a, and decrease the power consumption speed of the smart collar 1a so as to increase the power capacity or extend the use time of the smart collar 1a.

As shown in FIG. 2C, the components and their relations of the smart collar 1b of FIG. 2C are the same as those of the smart collar 1a. Different from the smart collar 1a, the smart collar 1b includes a rotatable block and a non-rotatable block. The main body 11 and other components (including the image capturing module 13) disposed in the main body 11 within the rotatable block are isolated and rotatable with respect to other blocks. Accordingly, the image capturing module 13 can be rotated along with the main body 11 in different angles. In other words, when the user utilizes the record function or the camera function of the smart collar 1b, the image capturing module 13 can be adjusted or rotated by the user to a desired angle for retrieving the images or screens in different angles. This configuration allows the smart collar 1b to have a wider viewing angle, so it can take the photo or image in front of the user and in other angles.

The other technical features of the smart collar 1b can be referred to the smart collar 1a, so the detailed description thereof will be omitted.

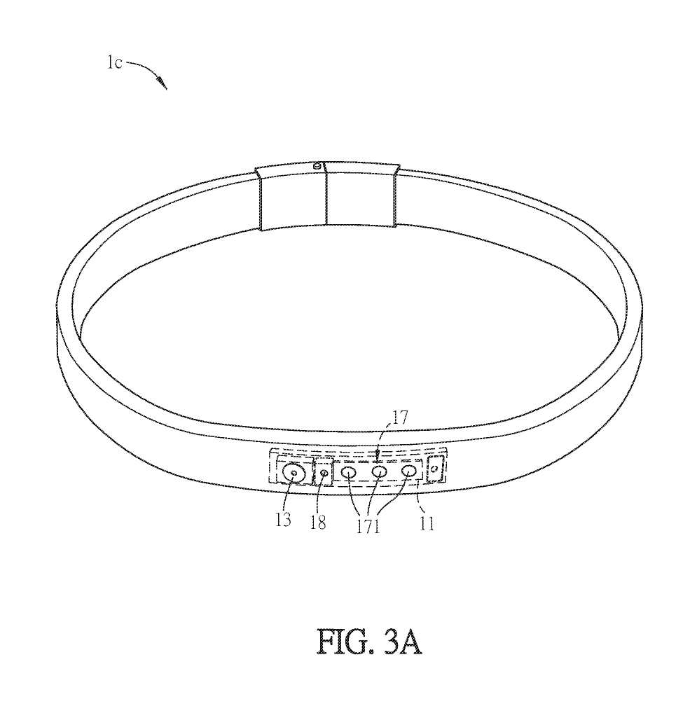

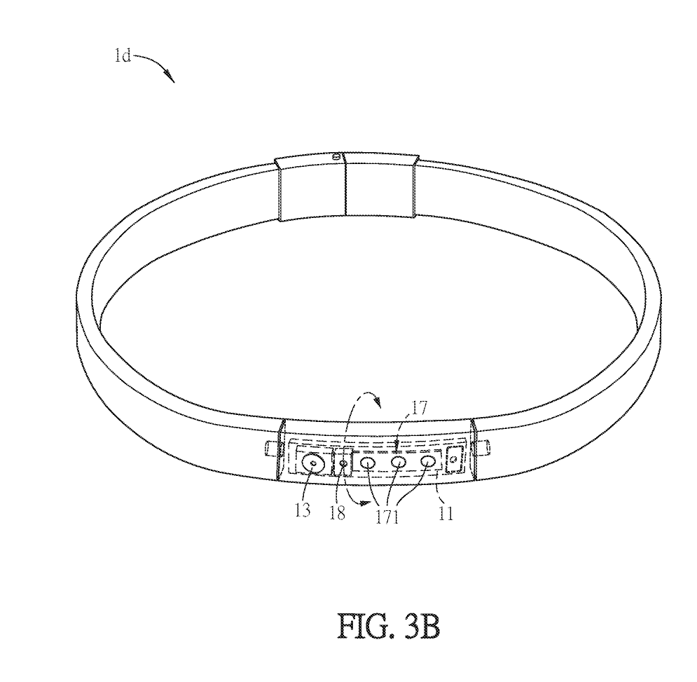

FIG. 3A is a schematic diagram of a smart collar 1c according to another embodiment of the disclosure. As shown in FIG. 3A, the components and their relations of the smart collar 1c are mostly the same as those of the smart collar 1a. Different from the smart collar 1a, the smart collar 1c further includes a voice retrieving module 18 coupled to the power module 12 and the process-storage module 15. The voice retrieving module 18 retrieves at least one voice data, and the storage unit 152 further stores the voice data. The voice retrieving module 18 is preferably an MEMS microphone. In this embodiment, the smart collar 1c includes one MEMS microphone. In other aspects, the smart collar 1c may include two or more MEMS microphones (e.g. 5 MEMS microphones). Besides, the ANC (Active Noise Cancellation) technology can be used to erase the undesired noise so as to improve the retrieved voice signal. When the user wears the smart collar and stays in a noise or crowd environment, the configuration of multiple MEMS microphones can help the voice retrieving module to retrieve the pour and clear voice data with good quality.

Accordingly, the smart collar 1c can retrieve the image data and the voice data by the image capturing module 13 in cooperated with the voice retrieving module 18, so that the smart collar 1c can comprehensively record the procedure and other information details in the exercise or sport of the user.

Similarly, as shown in FIG. 3B, the components and their relations of the smart collar 1d of FIG. 3B are the same as those of the smart collar 1c. Different from the smart collar 1c, the smart collar 1d includes a rotatable block and a non-rotatable block. The main body 11 and other components (including the image capturing module 13 and the voice retrieving module 18) disposed in the main body 11 within the rotatable block are isolated and rotatable with respect to other blocks.

Accordingly, the image capturing module 13 and the voice retrieving module 18 can be rotated along with the main body 11 in different angles with reference to other blocks. In other words, when the user utilizes the record function, the camera function or the voice retrieving function of the smart collar 1d, the image capturing module 13 and the voice retrieving module 18 can be adjusted or rotated by the user to a desired angle for retrieving the images or screens in different angles. This configuration allows the smart collar 1d to have a wider viewing angle, so it can take the image or retrieve the voice in front of the user and in other angles.

The other technical features of the smart collar 1d can be referred to the smart collar 1c, so the detailed description thereof will be omitted.

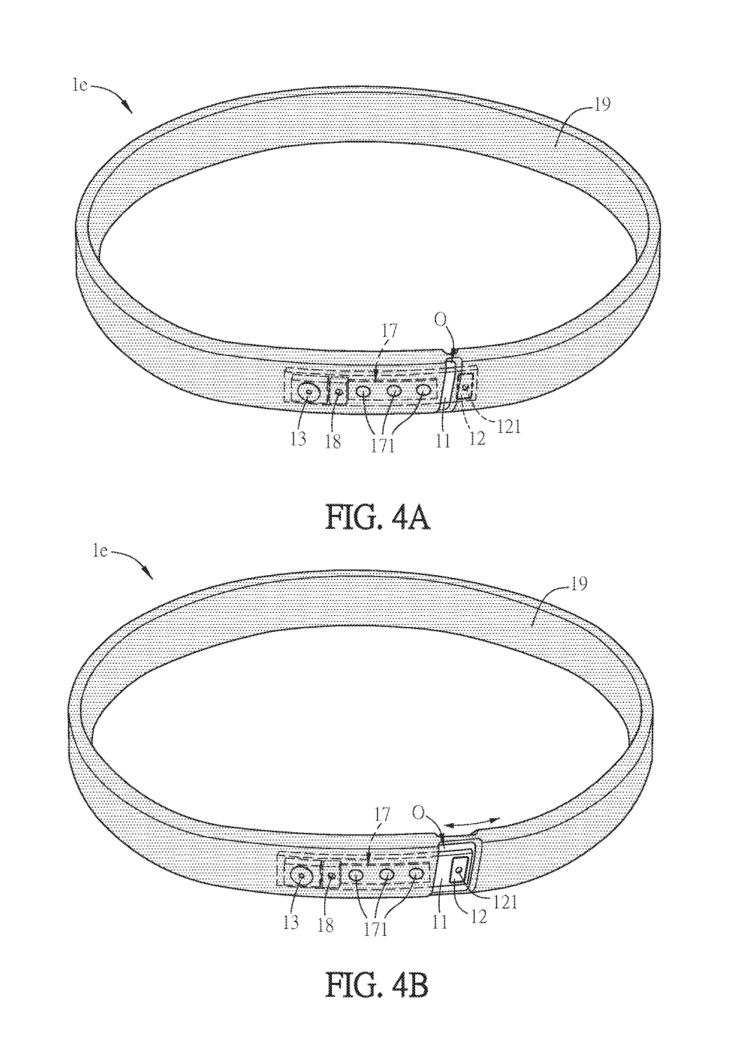

FIG. 4A is a schematic diagram of a smart collar, which has a protection body, according to another embodiment of the disclosure, and FIG. 4B is a schematic diagram of the smart collar of FIG. 4A as the opening of the protection body is exposed.

As shown in FIGS. 4A and 4B, the components of the smart collar 1e of FIG. 3B this embodiment are mostly the same as those of the smart collar 1a. Different from smart collar 1a, the main body 11 of the smart collar 1e is covered by a protection body 19. In practice, the protection body 19 can be designed as the casing of the smart collar 1e or designed to directly contact with and fix to the user's body. In order to provide a comfortable wearing experience, the smart collar 1e should be fit to the user's body, and the material of the smart collar 1e is properly selected to prevent the undesired allergies and itches. For example, the material of the protection body 19 can be rubber or silica gel, so that the user can comfortably wear the smart collar.

In practice, the protection body 19 of FIG. 4A is an extendable elastic body.

In other words, the protection body 19 is made of extendable rubber or silica gel. The protection body 19 has an opening O, and the opening O is disposed outside the main body 11. When the user opens the opening O of the extendable elastic body, the main body 11 is not exposed from the opening O. In addition, since the smart collar 1e is made of the extendable elastic rubber or silica gel, the smart collar 1e can flexibly fixed to the user's body or neck without the hook structure of the smart collar 1a.

When the extendable elastic body is stretched, as shown in FIG. 4B, the main body 11 is exposed from the extendable elastic body via the opening O, and one or a combination of the power module, the image capturing module, the sensing module and the process-storage module of the smart collar 1e, which are disposed in the main body 11, is adjusted to control the smart collar 1e to switch from a first configuration to a second configuration. To be noted, in this example, the power module 12 is exposed from the main body 11 via the opening O, so that the user can replace, increase or decrease the battery 122. Similarly, in other aspects, another module can be exposed from the main body 11 via the opening O, so that the user can replace, increase or decrease the component in the exposed module.

In order to replace the battery 122, the user can take the used battery with lower power or bad charging efficiency off, and then put on the new battery with higher power or normal charging efficiency. In other words, when the battery of the smart collar 1e is in a lower power status or has poor charging efficiency, the smart collar 1e is in a first configuration; when the battery of the smart collar 1e is in a high power status or has normal charging efficiency, the smart collar 1e is in a second configuration.

In order to increase the battery 122, the user can increase the battery 122 of the smart collar 1e from one battery 122 to two batteries 122. This configuration can increase the power capacity, use time and standby time of the smart collar 1e. In this case, when the smart collar 1e has one battery 122, the smart collar 1e is in a first configuration; when the smart collar 1e has two batteries 122, the smart collar 1e is in a second configuration.

Accordingly, when the extendable elastic body is stretched, the opening can be exposed from the extendable elastic body, and then the user can adjust one or a combination of the power module, the image capturing module, the sensing module and the process-storage module to control the smart collar to switch from a first configuration to a second configuration. This operation can simply improve the function and expand the application of the smart collar.

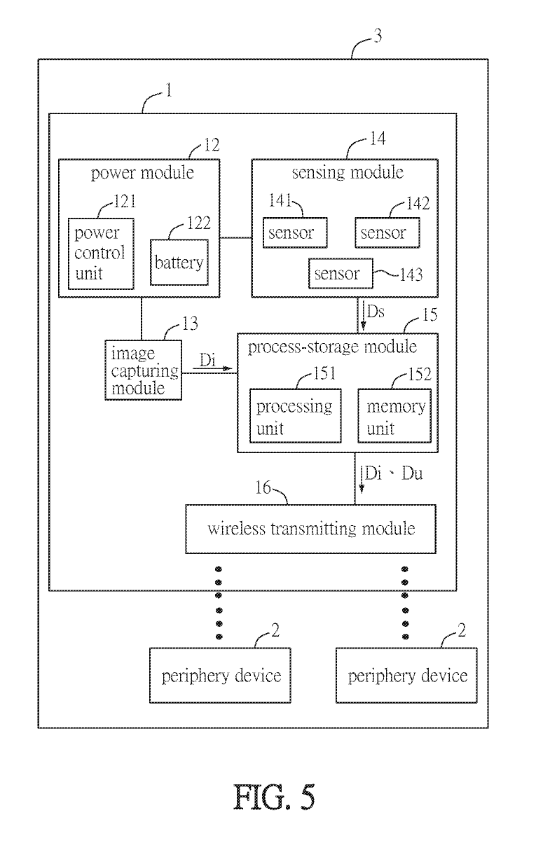

FIG. 5 is a block diagram of a smart collar system according to an embodiment of the disclosure. As shown in FIG. 5, the smart collar system includes at least one periphery device and a smart collar. In this embodiment, the smart collar system 3 includes one smart collar 1 and two periphery devices 2. Of course, in other embodiments, the smart collar 1 of the smart collar system 3 can be communicated with three or more periphery devices, and this disclosure is not limited.

Moreover, the smart collar 1 can be communicated with two periphery devices 2 via the wireless transmitting module 16, and the wireless transmitting module 16 transmits the user condition data Du to the two peripheral devices 2. This configuration can carry out the dual-directional data transmission between the smart collar 1 and the periphery devices 2. The technical features of the smart collar 1 can be referred to the above embodiments, so the detailed description thereof will be omitted.

In summary, the smart collar of the disclosure is configured to detecting the physical condition, motion condition and environment condition of a user so as to retrieve the user condition data. The smart collar includes a main body, a power module, an image capturing module, a sensing module, a process-storage module, and a wireless transmitting module. Thus, the smart collar has good power capacity, use time and standby time, and the user does not need to recharge the smart collar frequently. Besides, the smart collar can record the detailed sport events and retrieve other information.

In addition, the smart collar of the disclosure can transmit the image data and user condition data to the peripheral device through the wireless transmitting module. Moreover, the main body can integrate with all modules, and the smart collar can be bent to fit the human body without affecting the functions of the modules.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. A smart collar configured for detecting a physical condition, a motion condition and an environment condition of a user for retrieving user condition data, the smart collar comprising:
a main body;
a power module disposed in the main body and comprising a power control unit and a battery;
an image capturing module coupled to the power module and retrieving at least one image data;
a sensing module coupled to the power module and comprising at least a sensor for detecting at least one physical condition, at least one motion condition and at least one environment condition of the user so as to obtain physical condition data, motion condition data and environment condition data;
a process-storage module coupled to the image capturing module and the sensing module and comprising at least a processing unit and at least a memory unit, wherein the memory unit is coupled to the processing unit, and the processing unit generates the user condition data according to the physical condition data, the motion condition data and the environment condition data, and the memory unit stores the image data and the user condition data; and
a wireless transmitting module communicated with a peripheral device and transmitting the image data and the user condition data to the peripheral device.

2. The smart collar of claim 1, further comprising:
a voice retrieving module coupled to the power module and the process-storage module and retrieving at least one voice data, wherein the storage unit further stores the voice data.

3. The smart collar of claim 1, further comprising:
a display module disposed in the main body and coupled to the process-storage module.

4. The smart collar of claim 3, wherein the display module is a lamp display module.

5. The smart collar of claim 1, further comprising:
a protection body covering the main body.

6. The smart collar of claim 5, wherein the protection body is made of rubber or silica gel.

7. The smart collar of claim 5, wherein the protection body is an extendable elastic body having an opening, and the opening is disposed outside the main body.

8. The smart collar of claim 7, wherein when the extendable elastic body is stretched, the main body is exposed from the extendable elastic body via the opening, and one or a combination of the power module, the image capturing module, the sensing module and the process-storage module is adjusted to control the smart collar to switch from a first configuration to a second configuration.

9. The smart collar of claim 1, wherein the sensor is one or a combination of an optical heart rate sensor, a reflective pulse oximeter, a multi-axis gyroscope sensor, a multi-axis acceleration sensor, a geomagnetism sensor, an inertial sensor, a thermal sensor, an air pressure sensor, a humidity sensor, a pressure sensor, a height sensor, a light sensor, and a gas sensor.

10. The smart collar of claim 1, wherein the user condition data comprises one or a combination of heart rate information, pulse information, Blood oxygen information, emotion information, sleep infoitnation, step infoiiiiation, distance information, caloric consumption information, body information, sitting time information, temperature information, air pressure information, humidity information, pressure information, height information, light information, and gas information.

11. A smart collar system, comprising:
at least a peripheral device; and
a smart collar configured for detecting a physical condition, a motion condition and an environment condition of a user, wherein the smart collar comprises:
a main body;
a power module disposed in the main body and comprising a power control unit and a battery;
an image capturing module coupled to the power module and retrieving at least one image data;
a sensing module coupled to the power module and comprising at least a sensor for detecting at least one physical condition, at least one motion condition and at least one environment condition of the user so as to obtain physical condition data, motion condition data and environment condition data;
a process-storage module coupled to the image capturing module and the sensing module and comprising at least a processing unit and at least a memory unit, wherein the memory unit is coupled to the processing unit, and the processing unit generates the user condition data according to the physical condition data, the motion condition data and the environment condition data, and the memory unit stores the image data and the user condition data; and
a wireless transmitting module communicated with the peripheral device and transmitting the image data and the user condition data to the peripheral device.

12. The smart collar system of claim 11, wherein the smart collar further comprises:
a voice retrieving module coupled to the power module and the process-storage module and retrieving at least one voice data, wherein the storage unit further stores the voice data.

13. The smart collar system of claim 11, wherein the smart collar further comprises:
a display module disposed in the main body and coupled to the process-storage module.

14. The smart collar system of claim 13, wherein the display module is a lamp display module.

15. The smart collar system of claim 11, wherein the smart collar further comprises:
a protection body covering the main body.

16. The smart collar system of claim 15, wherein the protection body is made of rubber or silica gel.

17. The smart collar system of claim 15, wherein the protection body is an extendable elastic body having an opening, and the opening is disposed outside the main body.

18. The smart collar system of claim 17, wherein when the extendable elastic body is stretched, the main body is exposed from the extendable elastic body via the opening, and one or a combination of the power module, the image capturing module, the sensing module and the process-storage module is adjusted to control the smart collar to switch from a first configuration to a second configuration.

19. The smart collar system of claim 11, wherein the sensor is one or a combination of an optical heart rate sensor, a reflective pulse oximeter, a multi-axis gyroscope sensor, a multi-axis acceleration sensor, a geomagnetism sensor, an inertial sensor, a thermal sensor, an air pressure sensor, a humidity sensor, a pressure sensor, a height sensor, a light sensor, and a gas sensor.

20. The smart collar system of claim 11, wherein the user condition data comprises one or a combination of heart rate information, pulse infoi nation, Blood oxygen information, emotion information, sleep information, step information, distance information, caloric consumption information, body information, sitting time information, temperature information, air pressure information, humidity information, pressure information, height information, light information, and gas information.
